(19) United States
(12) Patent Application Publication
(10) Pub. No.: US 2023/0038074 A1
(43) Pub. Date: Feb. 9, 2023

(54) MAGNETIC MINIATURE DEVICE AND SYSTEM FOR REMOTELY MANEUVERING IT

(71) Applicant: BIONAUT LABS LTD., Herzliya (IL)

(72) Inventors: Florent CROS, Los Angeles, CA (US); Michael SHPIGELMACHER, Los Angeles, CA (US); Alex KISELYOV, San Diego, CA (US); Darrell HARRINGTON, Canoga Park, CA (US); Suehyun CHO, Los Angeles, CA (US); Hovhannes SARGSYAN, North Hollywood, CA (US)

(73) Assignee: BIONAUT LABS LTD., Herzliya (IL)

(21) Appl. No.: 17/784,329

(22) PCT Filed: Dec. 16, 2020
(86) PCT No.: PCT/US2020/065207

§ 371 Date: Jun. 10, 2022

Related U.S. Application Data
(60) Provisional application No. 62/948,472, filed on Dec. 16, 2019.

(51) Int. Cl. A61M 31/00; A61M 25/01; A61K 9/48

(52) U.S. Cl. CPC A61M 31/002; A61K 9/4808; A61M 25/0127

(57) ABSTRACT

A miniature device configured to be maneuvered within a patient under manipulation by an external magnetic field and to selectively perform a predefined function is provided. The miniature device comprises a shell defining therewithin an internal cavity, and a magnetic arrangement disposed within the cavity. The miniature device is configured such that the magnetic arrangement, within a rotating magnetic field, effects one of performance of the function and propulsion of the miniature device within the patient, and, within a magnetic field gradient, effects the other of performance of the function and propulsion of the miniature device within the patient.

### FIELD OF THE INVENTION

The presently disclosed subject matter relates to systems and miniature device configured to navigate within a patient to deliver a payload to a predetermined location therewithin, and in particular to such systems which use magnetic fields to direct operation of miniature devices within a patient.

### BACKGROUND

Remote control of medical devices moving inside the human body can be useful for a variety of purposes, including delivery of therapeutic payloads, diagnostics or surgical procedures. Such devices may include microscale or nanoscale robots, medical tools, "smart pills," etc. Such devices may be able to move in the body either through self-propulsion or an external propulsion mechanism. Accurate location and tracking of such devices may be necessary to ensure their proper functioning at the right anatomical location, and more specifically accurate delivery of the therapeutic payloads and/or diagnostics substances.

### SUMMARY

According to an aspect of the presently disclosed subject matter, there is provided a miniature device configured to be maneuvered within a patient under manipulation by an external magnetic field and to selectively perform a predefined function, the miniature device comprising a shell defining therewithin an internal cavity, and a magnetic arrangement disposed within the cavity,

The miniature device being configured such that the magnetic arrangement, within a rotating magnetic field, effects one of performance of the function and propulsion of the miniature device within the patient, and, within a magnetic field gradient, effects the other of performance of the function and propulsion of the miniature device within the patient.

The miniature device may further comprise a payload, wherein the performance of the function comprises release of the payload.

The miniature device may further comprise a payload, wherein the performance of the function comprises activating the tool.

The miniature device may be configured such that the magnetic arrangement, within a rotating magnetic field, effects the propulsion of the miniature device within the patient, and, within a magnetic field gradient, effects the performance of the function.

The shell may be formed as an elongated member extending along a drive axis and comprising a drive thread formed on an exterior surface thereof helically about the drive axis, the magnetic arrangement comprising a propulsion magnet rigidly connected to an interior surface thereof and disposed such that the vector of its magnetic moment, i.e., the orientation of its north and south poles, is disposed transverse (e.g., substantially perpendicular) to the drive axis.

The drive thread may comprise a tapered portion on a front end of the miniature device.

The magnetic arrangement may further comprise an ejection magnet slidably disposable, e.g., by a magnetic field gradient, within the internal cavity and an outlet, the payload being disposed between the ejection magnet and the outlet.

The miniature may further comprise a spacer disposed between the drive magnet and the ejection magnet.

The miniature device may be configured such that the magnetic arrangement, within a magnetic field gradient, effects the propulsion of the miniature device within the patient, and, within a rotating magnetic field, effects the performance of the function.

The shell may be formed as an elongated member extending along a drive axis between front and rear ends, the miniature device further comprising an actuation mechanism configured to facilitate the performance of the function.

The actuation mechanism may be further configured to facilitate the propulsion of the miniature device within the patient.

The actuation mechanism may comprise:
a linear gear on an interior surface of the shell; and
a pinion gear having a rotation axis transverse to the drive axis and being meshed to the linear gear, the pinion gear comprising a magnet of the magnetic arrangement rigidly coupled thereto such that the vector of its magnetic moment is disposed transverse to the rotation axis of the pinion gear, wherein rotation of the pinion gear about its rotation axis causes relative linear motion between it and the linear gear.

The coupling of the pinion gear to the magnet may comprise attachment of the magnet to a non-magnetic pinion gear that moves therewith, the magnet being formed as the pinion gear, etc., provided that an application of a rotational magnetic field results in rotation of the pinion gear.

The miniature device may further comprise an outlet, the payload being disposed between the pinion gear and the outlet, i.e., movement of the pinion gear pushes the payload towards the outlet and ejects it thereby.

The actuation mechanism may further comprise an ejection member coupled to the pinion gear to move generally linearly therewith along the drive axis. The actuation mechanism may further comprise a spring element configured to bias the ejection member toward the outlet.

The shell may comprise first and second shell members moveable with respect to one another along the drive axis, the actuation mechanism comprising a first linear gear on an interior surface of the first shell member and a second linear gear on an interior surface of the second shell member, the pinion gear being meshed to the first and second linear gears,

Wherein rotation of the pinion gear causes relative linear motion between the first and second linear gears, e.g., along the drive axis, thereby at least partially separating the first and second shell members.

The payload may be disposed within the internal cavity, wherein separation of the shell members facilitates release of the payload therefrom.

The second shell member may be biodegradable and comprise the payload, the actuation mechanism being configured to facilitate separation of the second shell member from the first shell member and from the pinion gear.

The actuation mechanism may comprise:
a magnet of the magnetic arrangement disposed such that the vector of its magnetic moment is disposed transverse to the drive axis; and
an actuation member coupled to the magnet and being configured to effect the performance of the function when rotated about the drive axis.

The shell may comprise an outlet at one of the ends, the actuation member comprising a plane twisted generally helically about the drive axis, such that rotation of the actuation member about the drive axis impels the payload toward the outlet, thereby facilitating its release.

The miniature device may further comprise two actuation members coupled to the magnet on opposite sides thereof, the actuation members being twisted about the drive axis in opposite senses, the shell comprising an outlet at each of its ends.

The magnetic arrangement may comprise two of the magnets with the actuation member spanning therebetween.

Each of the of the magnets may constitute a portion of a support element having a pointed end, each of the pointed ends substantially contacting a portion of the interior surface of the shell opposite one of the front and rear ends thereof.

The miniature device may comprise an outlet formed in a sidewall of the shell, the actuation member comprising a plane twisted generally helically about the drive axis and spanning between the magnets.

The actuation mechanism may comprise:
a drive nut formed on an interior surface of the shell; and
a lead screw having a rotation axis parallel to the drive axis and being meshed to the drive nut, the lead screw comprising a magnet of the magnetic arrangement rigidly coupled thereto such that the vector of its magnetic moment is disposed transverse to the rotation axis of the lead screw, wherein rotation of the lead screw about its rotation axis causes relative linear motion between it and the drive nut.

The coupling of the lead screw to the magnet may comprise attachment of the magnet to a non-magnetic lead screw that moves therewith, the magnet being formed as the lead screw, etc., provided that an application of a rotational magnetic field results in rotation of the lead screw.

The miniature device may further comprise an outlet, the payload being disposed between the lead screw and the outlet.

The payload may constitute a portion of the shell, a portion of the drive nut being formed on an interior surface of the payload, wherein the lead screw is partially meshed to the portion of the drive nut formed on the interior surface of the payload

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 schematically illustrates a system according to the presently disclosed subject matter for remotely maneuvering a miniature device within a patient;

FIG. 2 is a cross-sectional view of an example of a miniature device of the system illustrated in FIG. 1;

FIG. 3A is a cross-sectional view of another example of a miniature device of the system illustrated in FIG. 1;

FIG. 3B is a cross-sectional view of a modification of the miniature device illustrated in FIG. 3A;

FIGS. 4A and 4B are cross-sectional views of another example of a miniature device of the system illustrated in FIG. 1, in closed and open positions, respectively;

FIG. 5A is a cross-sectional view of another example of a miniature device of the system illustrated in FIG. 1;

FIGS. 5B and 5C are cross-sectional views of modifications of the miniature device illustrated in FIG. 5A;

FIG. 6A is a cross-sectional view of another example of a miniature device of the system illustrated in FIG. 1; and

FIG. 6B is a cross-sectional view of a modification of the miniature device illustrated in FIG. 6A.

### DETAILED DESCRIPTION

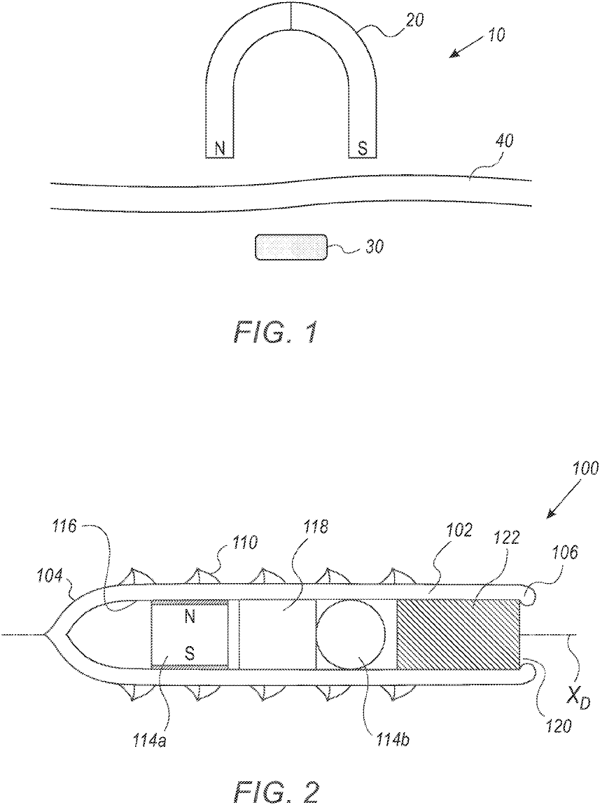

As illustrated in FIG. 1, there is provided a system, which is generally indicated at 10, for remotely maneuvering a magnetic device, in particular a miniature device, within a patient, and for performing a predefined function, e.g., releasing a payload and/or operating a tool at a predetermined location in the patient. They payload may comprise, e.g., one or more chemical compounds of medicinal, diagnostic, evaluative, and/or therapeutic relevance, one or more small molecules, biologics, cells, one or more radioisotopes, one or more vaccines, one or more mechanical devices, etc.

The system may be provided as described in any one or more of WO 19/213368, WO 19/213362, WO 19/213389, WO 20/014420, WO 20/092781, WO 20/092750, WO 18/204687, WO 18/222339, WO 18/222340, WO 19/212594, WO 19/005293, PCT/US20/58964, and PCT/US20/60677, the full contents of which are incorporated herein by reference.

The system 10 comprises a magnetic inducing apparatus 20 and a miniature device 30 carrying the payload and configured to be controlled by the magnetic inducing apparatus. It will be appreciated that while the following description will refer to a payload and/or delivery/release thereof, this is for convenience only and is not to be construed as limiting; the presently disclosed subject matter applies as well to a tool and/or delivery/operation thereof, and/or performance of any other predefined function, mutatis mutandis.

The magnetic inducing apparatus 20 is configured to be operated to selectively generate one or both of a rotating magnetic field and a magnetic field gradient. Application of these fields may be used to control the miniature device 30 within the patient 40, i.e., to maneuver it and to cause it to perform a predefined function. As will be described hereinbelow, the miniature device is configured such that it is maneuvered within one of these magnetic fields (i.e., either a rotating magnetic field or a magnetic field gradient), and caused to perform the predefined function under the other of these magnetic field.

The rotating magnetic field may be any magnetic field which induces a dipole magnet therewithin to spin, in particular (but not exclusively) about an axis which is substantially perpendicular to the magnetic vector of the magnet. Accordingly, the rotating magnetic field may be one produced by a magnet rotating about an axis perpendicular to its magnetic vector, a time-varying magnetic field, etc.

The magnetic field gradient may be any magnetic field in which a magnet therewithin moves. Accordingly, it may be a produced by one or more coils which operate to produce a varying magnetic field, by one or more magnets which are moved about the patient, etc.

According to some examples, the miniature device may be configured to be maneuvered by the rotating magnetic field, i.e., selective application and/or varying of a rotating magnetic field may be employed to propel the miniature device within the patient. Moreover, such a miniature device is configured to release a payload under direction a magnetic field gradient, i.e., selective application and/or varying of a magnetic field gradient may be employed to release a payload carried by the miniature device.

As illustrated in FIG. 2, the miniature device 100 may comprise a shell 102 being formed as an elongated member extending along a drive axis X_{D} extending between a front end 104 and a rear end 106 thereof. The shell 102 defines an internal cavity 108 therewithin. The front and/or rear end 104, 106 of the shell 102 may be tapered (as a non-limiting example, only the front end is shown as tapered in the accompanying figures). According to some examples, the drive thread 110 may be partially formed on the tapered portion of the shell 102.

The miniature device 100 further comprises a magnetic arrangement 114, configured to interact with externally applied magnetic fields, i.e., with a rotating magnetic field to effect propulsion of the miniature device, and with a magnetic field gradient to facilitate releasing the payload. Accordingly, the magnetic arrangement 114 comprises a propulsion magnet 114a and an ejection magnet 114b.

The propulsion magnet 114a is rigidly attached, i.e., it moves together with, an interior surface 116 of the shell 102, and is oriented such that the vector of its magnetic moment is disposed substantially transverse to the drive axis X_{D}, i.e., its north and south poles are adjacent opposite elongate sides of the interior surface 116, as indicated.

The ejection magnet 114b is free to slide within the cavity along the drive axis X_{D}. It may be spherical (as illustrated), or any other suitable shape which permits it to freely slide along the drive axis X_{D}, e.g., being cylindrical, etc. A spacer 118 is disposed between the propulsion magnet 114a and the ejection magnet 114b, and sized such that the magnetic formed between the two magnets do not inhibit operation of the miniature device (e.g., the amount of force required to move the ejection magnet 114b in a direction away from the rotation magnet 114a, as will be described below, is not so great as to dislodge the miniature robot from its location, etc.). Accordingly, the spacer 118 may be made of any suitable non-magnetic material (herein the specification and appended claims, the term "non-magnetic" includes those materials having measurable but negligible magnetic properties).

The miniature device 100 further comprises an outlet 120 formed at one end of the shell 102, for example the rear end 106 thereof. The size of the outlet 120 may be at least slightly smaller than the diameter of the ejection magnet 114b, thereby preventing it from exiting the internal cavity 108 of the shell 102 during use. A payload 122 is disposed between the ejection magnet 114b and the outlet 120. A cover (not illustrated), for example made of a biocompatible material such as paraffin, may be provided blocking the outlet 120, thereby preventing interaction between the payload 122 and the external surroundings in the patient before it is released.

During use, when a suitably oriented rotating magnetic field is applied to the miniature device 100, it causes the propulsion magnet 114a, and thus the shell 102 which is rigidly attached thereto, to rotate about the drive axis X_{D}. The interaction of the drive thread 110 with its external surroundings in the patient causes the miniature device to be propelled therein. According to some examples, the miniature device 100 may be suited to being propelled through soft tissue of the patient, for example within the liver, etc. It will be appreciated that the exterior of the miniature device, for example its size, the geometry of the drive thread 110, etc., may be designed to be suitable for use within a particular location and/or organ in the patient.

In order to release the payload 122, a magnetic field gradient is applied which urges the ejection magnet 114b toward the outlet 120, thereby pushing the payload through the outlet and releasing it into the patient at the location of the miniature device 100. As mentioned above, the outlet 120 may be designed so as to prevent releasing of the ejection magnet 114b. According to examples in which a cover is provided on the outlet 120, the linear movement of the ejection magnet 114b toward the outlet displaces and/or ruptures it, thereby allowing the payload 122 to interact with the external surroundings in the patient only when it is released as described above.

According to some examples, the miniature device may be configured to be maneuvered by the magnetic field gradient, i.e., selective application and/or varying of a magnetic field gradient may be employed to propel the miniature device within the patient. Moreover, such a miniature device is configured to release a payload under direction a rotating magnetic field, i.e., selective application and/or varying of a rotating magnetic field may be employed to release a payload carried by the miniature device.

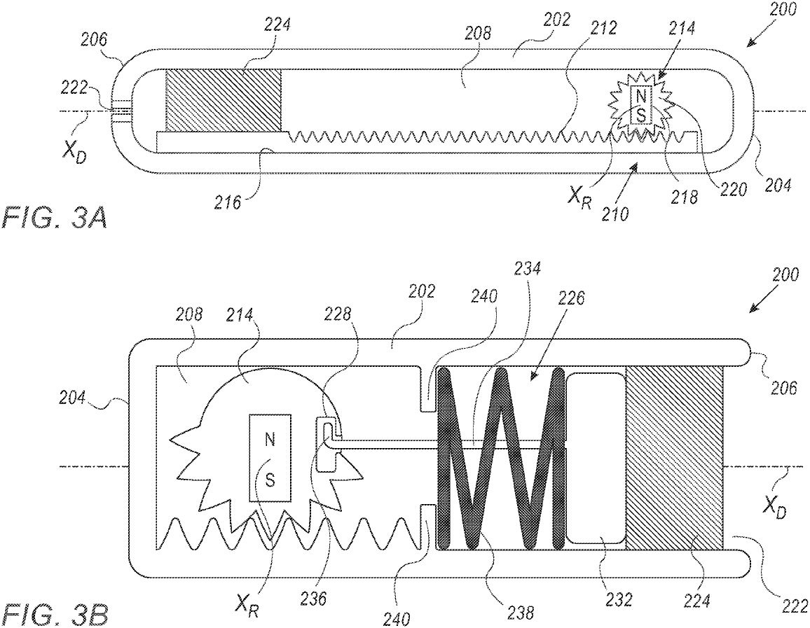

As illustrated in FIG. 3A, the miniature device 200 may comprise a shell 202 being formed as an elongated member extending along a drive axis X_{D} extending between a front end 204 and a rear end 206 thereof. The shell 202 defines an internal cavity 208 therewithin. An actuation mechanism, generally indicated at 210, is provided within the shell 202, configured to facilitate releasing the payload. It may be further configured to facilitate propulsion of the miniature device 200 within the patient, as will be described hereinbelow.

According to some examples, the actuation mechanism 210 comprises a linear gear (i.e., a rack) 212 and a pinion gear 214. The linear gear 212 is disposed on the interior surface 216 of the shell 202, for example extending parallel to the drive axis X_{D}.

The interior surface 216 of the shell 202 may be formed with suitable teeth constituting the linear gear 212, or a separate element comprising the linear gear may be attached to the interior surface.

The pinion gear 214 is disposed within the internal cavity 208 such that it's meshed to the linear gear 212, and arranged so that its rotation axis X_{R} is transverse to, e.g., substantially perpendicular to, the drive axis X_{D} of the miniature device 200. It further comprises a magnet 218 rigidly coupled thereto, such that the pinion gear 214 moves with the magnet. This may be accomplished, for example as shown such that the magnet 218 is received within a socket formed in a non-magnetic gear housing 220. Alternatively, the magnet 218 may constitute the pinion gear 214.

The magnet 218 is arranged such that the vector of its magnetic moment is disposed substantially transverse to the rotation axis X_{R} of the pinion gear 214, i.e., its north and south poles are adjacent opposite sides of the circumference of the pinion gear, as indicated.

The miniature device 200 further comprises an outlet 222 formed at one end of the shell 202, for example the rear end 206 thereof. The size of the outlet 222 may be at least slightly smaller than the diameter of the pinion gear 214, thereby preventing it from exiting the internal cavity 208 of the shell 202 during use. A payload 224 is disposed between the pinion gear 214 and the outlet 222. A cover (not illustrated), for example made of a biocompatible material such as paraffin, may be provided blocking the outlet 222, thereby preventing interaction between the payload 224 and the external surroundings in the patient before it is released.

During use, when a suitably oriented magnetic field gradient is applied to the miniature device 200, it drags the magnet 218, thereby propelling the miniature device within the patient.

In order to release the payload 224, a rotating magnetic field is applied, causing the magnet 218, and thus the pinion gear 214, to rotate about the rotation axis X_{R}, thereby causing it to move along the drive axis X_{D}, as a result of its meshing to the linear gear 212. When rotated such in a direction such that it moves towards the outlet 222, it pushes the payload 224 towards and through the outlet, thereby releasing it into the patient at the location of the miniature device 200. According to examples in which a cover is provided on the outlet 222, it is displaced and/or ruptured, thereby allowing the payload 224 to interact with the external surroundings in the patient only when it is released as described above.

As illustrated in FIG. 3B, the miniature device 200 may comprise a modified actuation mechanism 210, which further comprises an ejection member 226 coupled to the pinion gear 214, such that they move substantially in tandem along the drive axis X_{D}. According to some examples, the pinion gear 214 may comprise a notch 228 having an opening 230 with a reduced diameter. The ejection member 226 may be formed as a piston, with a head 232 being disposed adjacent the payload 224, and a shaft 234 thereof being formed with a hook 236 retained within the notch 228 formed in the pinion gear 214.

The actuation mechanism may further comprise a spring element 238, configured to bias the ejection member 226 toward the payload 224 in the direction of the outlet 222. The spring element 238 bears upon the head 232 of the ejection member 226 on one side, and against, e.g., projections 240 formed on the interior surface 216 of the shell 202, on an opposite side. The receipt of the hook 236 of the shaft 234 of the ejection member 226 within the notch 228 formed in the pinion gear 214 controls the extent to which the ejection member 226 moves along the drive axis X_{D} toward the outlet 222.

It will be appreciated that the term "piston" as used herein is descriptive of the general shape of the ejection member, and is not to be understood as limiting to features which are typical of pistons but unnecessary according to the presently disclosed subject matter, including, but not limited to, fluid-tightness, etc., mutatis mutandis.

In use, a suitably oriented magnetic field gradient may be applied to the miniature device 200 in order to propel it within the patient, for example as described above with reference to FIG. 3A.

In order to release the payload 224, a rotating magnetic field is applied, causing the magnet 218, and thus the pinion gear 214, to rotate about the rotation axis X_{R}, thereby causing it to move along the drive axis X_{D}, as a result of its meshing to the linear gear 212. When rotated such in a direction such that it moves towards the outlet 222, the bearing of the spring element 238 against the head 232 of the ejection member 226 pushes it, and thus the payload 224, towards and through the outlet, thereby releasing it into the patient at the location of the miniature device 200. According to examples in which a cover is provided on the outlet 222, it is displaced and/or ruptured, thereby allowing the payload 224 to interact with the external surroundings in the patient only when it is released as described above.

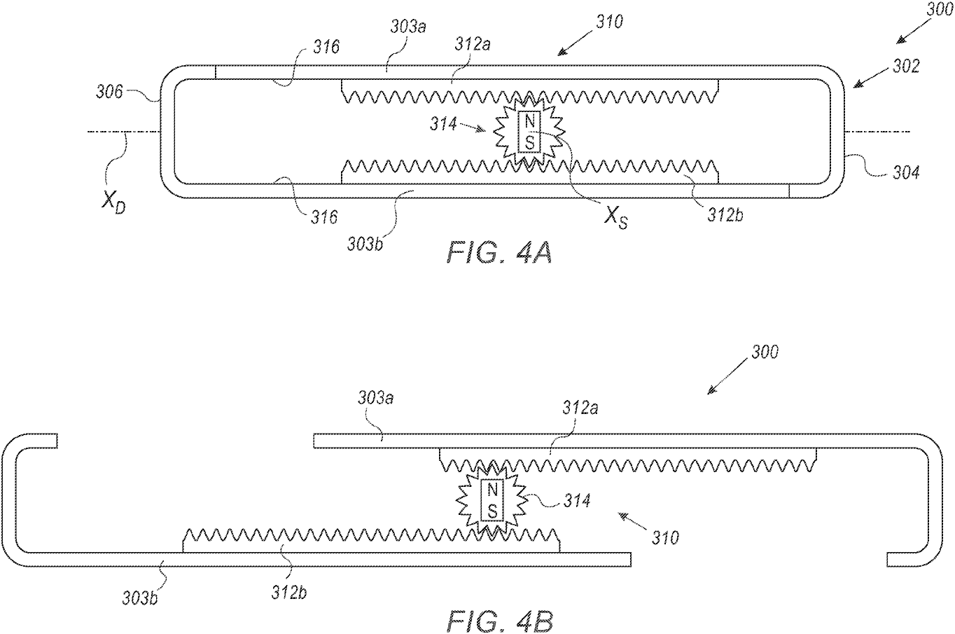

As illustrated in FIGS. 4A and 4B, the miniature device 300 may comprise a shell 302 comprising first and second shell members 303a, 303b, and which is formed as an elongated member extending along a drive axis X_{D} extending between a front end 304 and a rear end 306 thereof. (Herein the specification and appended claims, similar elements designated by a base reference numeral and a trailing letter may be collectively designed by a generic form of the element name and/or collectively designated by the base reference numeral along; for example, the first and second shell members 303a, 303b may herein be collectively referred to as "shell members" and/or collectively designated using reference numeral 303.) The shell members 303 may be configured to move with respect to one another along the drive axis X_{D}. The shell 302 defines an internal cavity 308 therewithin, in which a payload (not illustrated) is disposed. An actuation mechanism, generally indicated at 310, is provided within the shell 302, configured to facilitate releasing the payload. It may be further configured to facilitate propulsion of the miniature device 300 within the patient, as will be described hereinbelow.

The actuation mechanism 310 may comprise first and second linear gears 312a, 312b, and a pinion gear 314. Each of the linear gears 312a, 312b is disposed on the interior surface 316a, 316b of one of the shell members 303a, 303b, for example extending parallel to the drive axis X_{D}. The interior surfaces 316 may be formed with suitable teeth constituting the linear gears 312, or a separate element comprising the linear gear may be attached to the interior surface.

The pinion gear 314 is disposed within the internal cavity 308 such that it's meshed to the linear gears 312a, 312b, and arranged so that its rotation axis X_{R} is transverse to, e.g., substantially perpendicular to, the drive axis X_{D} of the miniature device 300. It further comprises a magnet 318 rigidly coupled thereto, such that the pinion gear 314 moves therewith. This may be accomplished, for example as shown such that the magnet 318 is received within a socket formed in a non-magnetic gear housing 320. Alternatively, the magnet 318 may constitute the pinion gear 314.

The magnet 318 is arranged such that the vector of its magnetic moment is disposed substantially transverse to the rotation axis X_{R} of the pinion gear 314, i.e., its north and south poles are adjacent opposite sides of the circumference of the pinion gear, as indicated.

During use, when a suitably oriented magnetic field gradient is applied to the miniature device 300, it drags the magnet 318, thereby propelling the miniature device within the patient.

In order to release the payload, a rotating magnetic field is applied, causing the magnet 318, and thus the pinion gear 314, to rotate about the rotation axis X_{R}, thereby causing the linear gears 312a, 312b to move along the drive axis X_{D} in opposite directions. This causes the shell members 303a, 303b to separate, i.e., from the closed position illustrated in FIG. 4A to the open position illustrated in FIG. 4B, thereby releasing the payload from the internal cavity 308 into the patient at the location of the miniature device 300.

According to a modification, one of the shell members 303a, 303b may be biodegradable and comprise the payload. According to some examples, it is itself the payload. According to such examples, the actuation mechanism 310 may be configured to effect separation of the first and second shell members 303a, 303b, thereby jettisoning the shell member comprising the payload, mutatis mutandis.

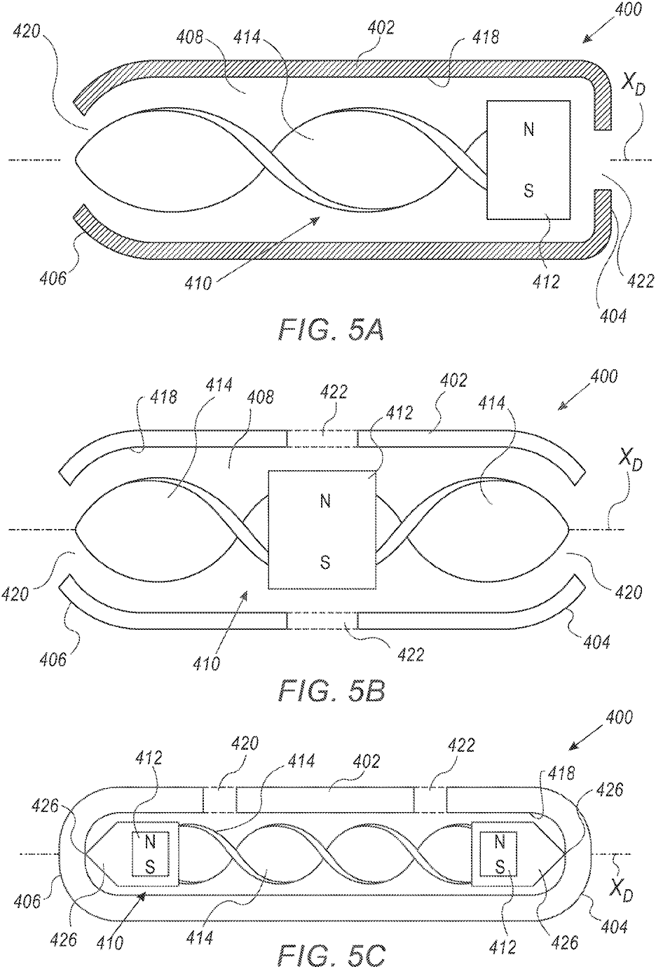

As illustrated in FIG. 5A, the miniature device 400 may comprise a shell 402 being formed as an elongated member extending along a drive axis X_{D} extending between a front end 404 and a rear end 406 thereof. The shell 402 defines an internal cavity 408 therewithin.

The miniature device 400 further comprises an actuation mechanism 410, comprising a magnet 412 and an actuation member 414 rigidly attached thereto, such that they move in tandem with one another. The actuation mechanism 410 is configured to interact with externally applied magnetic fields, i.e., with a magnetic field gradient to effect propulsion of the miniature device 400, and with a rotating magnetic field to facilitate releasing a payload (not illustrated) from within the internal cavity 408, as will be described below.

The magnet 412 is oriented such that the vector of its magnetic moment is disposed substantially transverse to the drive axis X_{D}, i.e., its north and south poles are adjacent opposite elongate sides of an interior surface 418 of the shell 402, as indicated.

The actuation member 414 may be formed of a plane twisted generally helically about the drive axis X_{D}, such that rotation thereof the actuation member about the drive axis impels the payload toward an outlet 420 formed in the shell 402, thereby facilitating its release. At the same time, the rotation of the actuation member 414 draws fluid from the environment surrounding the miniature device 400 into the internal cavity 408 of the shell 402 via an inlet 422.

As illustrated in FIG. 5B, according to some examples the actuation mechanism 410 comprises two actuation members 414, each disposed on an opposite side of the magnet 412, and each being twisted about the drive axis X_{D} in an opposite sense than in the other of the actuation members. The shell may comprise an outlet 420 at each of the front and rear ends 404, 406, and an inlet 422 in the middle of the shell 402, for example adjacent the magnet 412. Accordingly, the payload may be provided as two separate portions, each delivered via one of the outlets 420.

As illustrated in FIG. 5C, the actuation mechanism 410 may comprise two magnets 412 with the actuation member 414 spanning therebetween. Each of the of the magnets 412 may constitute a portion of a support element 424 having a pointed end 426, each of which substantially contacts a portion of the interior surface 418 of the shell 402 opposite one of the front and rear ends 404, 406 thereof. According to some examples, each of the support elements 424 comprises a housing 426 with one of the magnets 412 received therewithin; according to other examples, each magnet 412 constitutes one of the support elements 424.

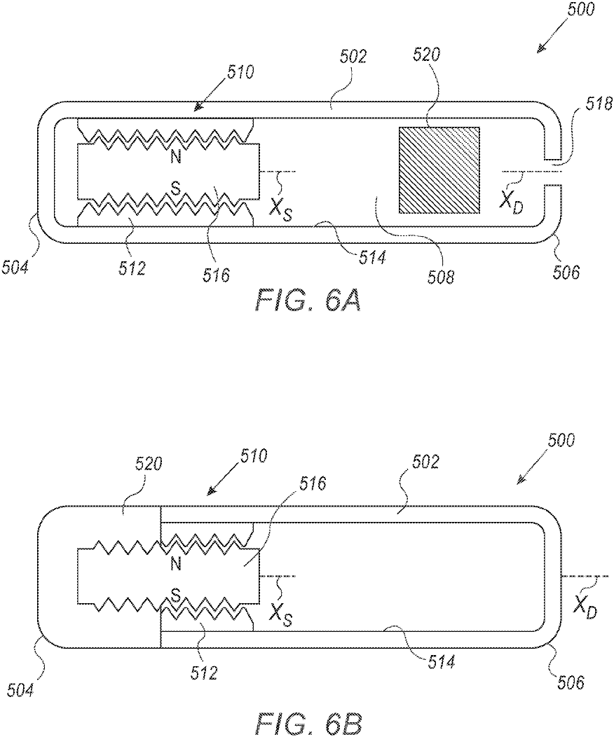

As illustrated in FIG. 6A, the miniature device 500 may comprise a shell 502 being formed as an elongated member extending along a drive axis X_{D} extending between a front end 504 and a rear end 506 thereof. The shell 502 defines an internal cavity 508 therewithin. An actuation mechanism, generally indicated at 510, is provided within the shell 502, configured to facilitate releasing the payload. It may be further configured to facilitate propulsion of the miniature device 500 within the patient, as will be described hereinbelow.

According to some examples, the actuation mechanism 510 comprises a drive nut 512 formed on an interior surface 514 of the shell 502, and a lead screw 516 having a rotation axis X_{R} parallel to the drive axis X_{D} of the shell 502 and being meshed to the drive nut.

The interior surface 514 of the shell 502, may be formed with suitable teeth constituting the drive nut 512, or a separate element comprising the drive nut may be attached to the interior surface. According to some examples, threads of the drive nut 512 and/or the lead screw 516 are only formed partway around the circumference of, respectively, the interior surface 514 of the shell 502 and/or the exterior of the lead screw, such that at any angular position of the lead screw with respect to the shell 502, the lead screw is sufficiently meshed to the drive nut, mutatis mutandis.

The lead screw 516 comprises a magnet rigidly coupled thereto. This may be accomplished, e.g., by providing a magnet which constitutes the lead screw 516 (as illustrated), or by providing a non-magnetic housing with a magnet received therein. The vector of the magnetic moment of the magnet is disposed transverse to the rotation axis X_{R} of the lead screw 516, i.e., its north and south poles are adjacent opposite sides of the circumference of the lead screw, as indicated. Accordingly, rotation of the lead screw 516 about its rotation axis X_{R} causes relative linear motion between it and the drive nut 512 along the drive axis.

The miniature device 500 further comprises an outlet 518 formed at one end of the shell 502, for example the rear end 506 thereof. The size of the outlet 518 may be at least slightly smaller than the diameter of the lead screw 516, thereby preventing it from exiting the internal cavity 508 of the shell 502 during use. A payload 520 is disposed between the lead screw 516 and the outlet 518. A cover (not illustrated), for example made of a biocompatible material such as paraffin, may be provided blocking the outlet 518, thereby preventing interaction between the payload 520 and the external surroundings in the patient before it is released.

During use, when a suitably oriented magnetic field gradient is applied to the miniature device 500, it drags the magnet associated with the lead screw 516 (i.e., either the lead screw itself, or a magnet constituting part of the lead screw), thereby propelling the miniature device within the patient.

In order to release the payload 520, a rotating magnetic field is applied, causing the lead screw 516, moving in tandem with its magnet, to rotate about the rotation axis X_{R}, thereby causing it to move along the drive axis X_{D}, as a result of its meshing to the drive nut 512. When rotated such in a direction such that it moves towards the outlet 518, it pushes the payload 520 towards and through the outlet, thereby releasing it into the patient at the location of the miniature device 500. According to examples in which a cover is provided on the outlet 518, it is displaced and/or ruptured, thereby allowing the payload 520 to interact with the external surroundings in the patient only when it is released as described above.

According to a modification, for example as illustrated in FIG. 6B, the payload 520 constitutes a portion of the shell 502. The drive nut 512 is formed partially on the payload 520. Accordingly, in order to release the payload 520, the lead screw 516 is rotated about its rotation axis X_{R} under a rotating magnetic field, as described above with reference to FIG. 6A, until it disengages with the payload 520.

According to any of the example disclosed above, internal friction may be reduced. Accordingly, any one or more of the following non-limiting examples may be employed to reduce the internal friction:
The interior surface of the shell may be formed so as to reduce friction. This may be accomplished by forming the shell such that the interior surface is highly smooth. According to some examples, for example in which portions of the actuation mechanism rotates about the drive axis X_{D}, circumferential micro-grooves may be formed on the interior surface of the shell.
The interior surface of the shell may be coated with a lubricant.
A ferrofluid may be provided in the internal cavity, for example to reduce friction around one or more of the magnets.
Materials of the components of the miniature device may be selected so as to lower friction therebetween.

According to any of the example disclosed above, external friction (i.e., between the miniature device and the surround environment within the patient) may be increased, for example to prevent rotation of the shell when components thereof are rotated therewithin. Accordingly, any one or more of the following non-limiting examples may be employed to increase the external friction:
An external magnetic field may be applied to bias the miniature device into an internal tissue of the patient, for example a soft tissue.
A non-lubricous coating may be applied to an exterior surface of the shell.
The exterior surface of the shell may be knurled.

It will be recognized that examples, embodiments, modifications, options, etc., described herein are to be construed as inclusive and non-limiting, i.e., two or more examples, etc., described separately herein are not to be construed as being mutually exclusive of one another or in any other way limiting, unless such is explicitly stated and/or is otherwise clear. Those skilled in the art to which this invention pertains will readily appreciate that numerous changes, variations, and modifications can be made without departing from the scope of the presently disclosed subject matter, mutatis mutandis.

1. A miniature device configured to be maneuvered within a patient under manipulation by an external magnetic field and to selectively perform a predefined function, the miniature device comprising a shell defining therewithin an internal cavity, and a magnetic arrangement disposed within the cavity,
said miniature device being configured such that the magnetic arrangement, within a rotating magnetic field, effects one of performance of the function and propulsion of the miniature device within the patient, and, within a magnetic field gradient, effects the other of performance of the function and propulsion of the miniature device within the patient.

2. The miniature device according to claim 1, further comprising a payload, wherein the performance of the function comprises release of said payload.

3. The miniature device according to claim 2, further comprising a tool, wherein the performance of the function comprises activating the tool.

4. The miniature device according to any one of claims 1 through 3, being configured such that the magnetic arrangement, within a rotating magnetic field, effects the propulsion of the miniature device within the patient, and, within a magnetic field gradient, effects the performance of the function.

5. The miniature device according to claim 4, said shell being formed as an elongated member extending along a drive axis and comprising a drive thread formed on an exterior surface thereof helically about the drive axis, said magnetic arrangement comprising a propulsion magnet rigidly connected to an interior surface thereof and disposed such that the vector of its magnetic moment is disposed transverse to the drive axis.

6. The miniature device according to claim 5, said drive thread comprising a tapered portion on a front end of the miniature device.

7. The miniature device according to any one of claims 4 through 6 when dependent on claim 2, the magnetic arrangement further comprising an ejection magnet slidably disposable within the internal cavity and an outlet, said payload being disposed between the ejection magnet and the outlet.

8. The miniature device according to claim 7, further comprising a spacer disposed between the drive magnet and the ejection magnet.

9. The miniature device according to any one of claims 1 through 3, being configured such that the magnetic arrangement, within a magnetic field gradient, effects the propulsion of the miniature device within the patient, and, within a rotating magnetic field, effects the performance of the function.

10. The miniature device according to claim 9, said shell being formed as an elongated member extending along a drive axis between front and rear ends, the miniature device further comprising an actuation mechanism configured to facilitate the performance of the function.

11. The miniature device according to claim 10, said actuation mechanism being further configured to facilitate the propulsion of the miniature device within the patient.

12. The miniature device according to any one of claims 10 and 11, said actuation mechanism comprising:
a linear gear on an interior surface of the shell; and
a pinion gear having a rotation axis transverse to the drive axis and being meshed to the linear gear, the pinion gear comprising a magnet of said magnetic arrangement rigidly coupled thereto such that the vector of its magnetic moment is disposed transverse to the rotation axis of the pinion gear, wherein rotation of the pinion gear about its rotation axis causes relative linear motion between it and the linear gear.

13. The miniature device according to claim 12 when dependent on claim 2, further comprising an outlet, said payload being disposed between the pinion gear and the outlet.

14. The miniature device according to claim 13, said actuation mechanism further comprising an ejection member coupled to the pinion gear to move generally linearly therewith along the drive axis.

15. The miniature device according to claim 14, said actuation mechanism further comprising a spring element configured to bias said ejection member toward the outlet.

16. The miniature device according to claim 12, the shell comprising first and second shell members moveable with respect to one another along the drive axis, said actuation mechanism comprising a first linear gear on an interior surface of the first shell member and a second linear gear on an interior surface of the second shell member, said pinion gear being meshed to the first and second linear gears,
wherein rotation of the pinion gear causes relative linear motion between the first and second linear gears, thereby at least partially separating said first and second shell members.

17. The miniature device according to claim 16 when dependent on claim 2, said payload being disposed within the internal cavity, wherein separation of the shell members facilitates release of the payload therefrom.

18. The miniature device according to any one of claims 16 and 17, wherein the second shell member is biodegradable and comprises the payload, the actuation mechanism being configured to facilitate separation of the second shell member from the first shell member and from the pinion gear.

19. The miniature device according to any one of claims 10 and 11, said actuation mechanism comprising:
a magnet of said magnetic arrangement disposed such that the vector of its magnetic moment is disposed transverse to the drive axis; and
an actuation member coupled to said magnet and being configured to effect the performance of the function when rotated about the drive axis.

20. The miniature device according to claim 19 when dependent on claim 2, the shell comprising an outlet at one of the ends, said actuation member comprising a plane twisted generally helically about the drive axis, such that rotation of the actuation member about the drive axis impels the payload toward the outlet, thereby facilitating its release.

21. The miniature device according to claim 20, further comprising two actuation members coupled to the magnet on opposite sides thereof, the actuation members being twisted about the drive axis in opposite senses, the shell comprising an outlet at each of its ends.

22. The miniature device according to claim 19 when dependent on claim 2, the magnetic arrangement comprising two of said magnets with the actuation member spanning therebetween.

23. The miniature device according to claim 21, wherein each of the of the magnets constitutes a portion of a support element having a pointed end, each of the pointed ends substantially contacting a portion of the interior surface of the shell opposite one of the front and rear ends thereof.

24. The miniature device according to any one of claims 22 and 23, comprising an outlet formed in a sidewall of the shell, said actuation member comprising a plane twisted generally helically about the drive axis and spanning between said magnets.

25. The miniature device according to any one of claims 10 and 11, said actuation mechanism comprising:
a drive nut formed on an interior surface of the shell; and
a lead screw having a rotation axis parallel to the drive axis and being meshed to the drive nut, the lead screw comprising a magnet of said magnetic arrangement rigidly coupled thereto such that the vector of its magnetic moment is disposed transverse to the rotation axis of the lead screw, wherein rotation of the lead screw about its rotation axis causes relative linear motion between it and the drive nut.

26. The miniature device according to claim 25 when dependent on claim 2, further comprising an outlet, said payload being disposed between the lead screw and the outlet.

27. The miniature device according to claim 25 when dependent on claim 2, wherein said payload constitutes a portion of the shell, a portion of said drive nut being formed on an interior surface of the payload, wherein the lead screw is partially meshed to the portion of the drive nut formed on the interior surface of the payload.
